(19) United States
(12) Patent Application Publication
(10) Pub. No.: US 2026/0083644 A1
(43) Pub. Date: Mar. 26, 2026

(54) METHOD FOR PRODUCING DENTAL POLYMERIZABLE COMPOSITION OR DENTAL POLYMERIZABLE COMPOSITION INTERMEDIATE

(71) Applicant: SHOFU INC., Kyoto-shi (JP)

(72) Inventors: Naoya KITADA, Kyoto-shi (JP); Rei NISHIMURA, Kyoto-shi (JP); Yusuke MIYASHIMA, Kyoto-shi (JP)

(21) Appl. No.: 19/336,762

(22) Filed: Sep. 23, 2025

(30) Foreign Application Priority Data
Sep. 25, 2024 (JP) 2024166482

(51) Int. Cl. A61K 6/887

(52) U.S. Cl. CPC A61K 6/887

(57) ABSTRACT

An object of the present invention is to provide a production method that can provide a dental polymerizable composition (β) or a dental polymerizable composition intermediate (γ), having excellent storage stability, in high yield. A method for producing a dental polymerizable composition (β) or a dental polymerizable composition intermediate (γ), which is substantially free of a filler, using a production machine that is substantially not equipped with a stirring blade and equipped with a container, the method comprising the steps of disposing an composition (α) comprising (a) a polymerizable monomer and (b) a polymerization inhibitor in the container such that an empty space rate of the container is 10 to 90%; and operating the container.

### BACKGROUND ART

### Technical Field

The present invention relates to a production method of a dental polymerizable composition or a dental polymerizable composition intermediate.

### Description of the Related Art

Conventionally, dental polymerizable compositions or dental polymerizable composition intermediates contain polymerizable monomers and polymerization inhibitors, and have been produced by mixing and stirring these components using a production machine with a stirring blade.

For example, in Patent Literature 1 (JP 2012-012314 A), a beaker that is an open-top container, is charged with materials, each component of which is mixed by stirring with a stirring blade without operating a stirring tank itself, to obtain a dental polymerizable composition.

However, the production method described in Patent Literature 1 has a problem in that when the stirring blade is removed from the mixture after completion of stirring, the mixture adheres to the stirring blade, resulting in a relatively poor yield.

Moreover, Patent Literature 2 (JP 2014-181183 A) discloses a method for producing a dental polymerizable composition using a planetary centrifugal mixer.

However, Patent Literature 2 relates to a method for homogeneously dispersing fumed silica, an inorganic dispersoid, in a dispersion medium containing a polymerizable monomer and/or an organic solvent using a planetary centrifugal mixer, and the literature does not disclose any method for producing a dental polymerizable composition or a dental polymerizable composition intermediate that does not contain fumed silica, and the aforementioned problems have not yet been solved.

### CITATION LIST

### Patent Literatures

Patent Literature 1: JP 2012-012314 A
Patent Literature 2: JP 2014-181183 A

### SUMMARY

An object of the present invention is to provide a production method that can provide a dental polymerizable composition (β) or a dental polymerizable composition intermediate (γ), having excellent storage stability, in high yield.

The present inventors have found as a result of diligent investigations that the aforementioned problems can be solved by a predetermined method for producing a dental polymerizable composition (β) or a dental polymerizable composition intermediate (γ).

### (Item 1)

A method for producing a dental polymerizable composition (β) or a dental polymerizable composition intermediate (γ), which is substantially free of a filler, using a production machine that is substantially not equipped with a stirring blade and equipped with a container, the method comprising the steps of: disposing a composition (α) comprising (a) a polymerizable monomer and (b) a polymerization inhibitor in the container such that an empty space rate of the container is 10 to 90%; and allowing the container to operate.

### (Item 2)

A method for producing a dental polymerizable composition (β) or a dental polymerizable composition intermediate (γ), which is substantially free of a filler, using a production machine that is substantially not equipped with a stirring blade and equipped with a container, the method comprising the steps of: disposing a composition (α) comprising (a) a polymerizable monomer and (b) a polymerization inhibitor in the container such that an empty space rate of the container is 10 to 90%; heating the container to between 30° C. and 70° C.; and allowing the container to operate.

### (Item 3)

The method for producing a dental polymerizable composition (β) or a dental polymerizable composition intermediate (γ) according to item 1 or 2, wherein in the production machine that is substantially not equipped with a stirring blade and equipped with a container, the container operates in one or more modes selected from rotation, hyperbolic motion, or vibration.

### (Item 4)

The method for producing a dental polymerizable composition (β) or a dental polymerizable composition intermediate (γ) according to item 1 or 2, wherein in the production machine that is substantially not equipped with a stirring blade and equipped with a container, the container operates in one or more modes selected from rotation at a speed of 60 min⁻¹ or less, hyperbolic motion at a speed of 50 min⁻¹ or less, or vibration at a speed of 400 min⁻¹ or less.

### (Item 5)

The method for producing a dental polymerizable composition (β) or a dental polymerizable composition intermediate (γ) according to any one of items 1 to 4, wherein the empty space rate of the container is 20 to 80%.

### (Item 6)

The method for producing a dental polymerizable composition (β) or a dental polymerizable composition intermediate (γ) according to any one of items 1 to 5, wherein the composition (α) comprises (c) an organic solvent.

### Effects of the Invention

The production method of the present invention can provide a dental polymerizable composition (β) or a dental polymerizable composition intermediate (γ), which has excellent storage stability and contains a composition (α) containing (a) a polymerizable monomer and (b) a polymerization inhibitor, in high yield.

### DETAILED DESCRIPTION OF THE PREFERRED EMBODIMENT(S)

The present invention discloses a method for producing a dental polymerizable composition (β) or a dental polymerizable composition intermediate (γ), which is substantially free of a filler, using a production machine that is substantially not equipped with a stirring blade and equipped with a container, the method comprising the steps of: disposing a composition (α) comprising (a) a polymerizable monomer and (b) a polymerization inhibitor in the container such that an empty space rate of the container is 10 to 90%; and allowing the container to operate. The production method of the present invention and the like will be described in detail below.

### <Dental Polymerizable Composition (β) or Dental Polymerizable Composition Intermediate (γ)>

The dental polymerizable composition (β) or the dental polymerizable composition intermediate (γ) in the present invention is a mixture that contains (a) a polymerizable monomer and (b) a polymerization inhibitor and is substantially free of a filler. Also, the dental polymerizable composition (β) or the dental polymerizable composition intermediate (γ) of the present invention preferably has a viscosity at 25° C. of 5,000 mPa-s or less. The dental polymerizable composition (β) having a viscosity of more than 5,000 mPa-s has poor dischargeable property from a storage container because of its high viscosity, and the dental polymerizable composition intermediate (γ) having a viscosity of more than 5,000 mPa-s is difficult to handle in the subsequent production step and is not practical.

A method for measuring a viscosity of the dental polymerizable composition (β) or the dental polymerizable composition intermediate (γ) can be employed without any limitations as long as it is a general analytical method. Examples thereof include a B-type viscometer, an E-type viscometer, and a dynamic viscoelasticity measurement apparatus.

### <(a) Polymerizable Monomer>

The (a) polymerizable monomer which is any publicly known polymerizable monomer having one or more polymerizable groups, can be used without any limitations. The polymerizable monomer is preferably a polymerizable monomer exhibiting radical polymerizability. From the viewpoint of facilitation of radical polymerization, the polymerizable group is preferably a radically polymerizable group and more preferably a (meth)acrylic group and/or a (meth)acrylamide group. As used herein, it is to be noted that the term “(meth)acrylic” refers to acrylic and/or methacrylic, the term “(meth)acryloyl” refers to acryloyl and/or methacryloyl, and the term “(meth)acrylate” refers to acrylate and/or methacrylate.

Examples of the (a) polymerizable monomers include one or more selected from a polymerizable monomer having no acidic group or no sulfur atom, (a1) a polymerizable monomer having an acidic group, or (a2) a polymerizable monomer having a sulfur atom.

Examples of the polymerizable monomer having no acidic group or no sulfur atom include one or more selected from a polymerizable monomer having one radically polymerizable group, a polymerizable monomer having two radically polymerizable groups, or a polymerizable monomer having three or more radically polymerizable groups.

Examples of the polymerizable monomer having one radically polymerizable group include one or more selected from 2-hydroxyethyl (meth)acrylate, 3-hydroxypropyl (meth)acrylate, 4-hydroxybutyl (meth)acrylate, 6-hydroxyhexyl (meth)acrylate, 10-hydroxydecyl (meth)acrylate, propylene glycol mono(meth)acrylate, glycerol mono(meth)acrylate, erythritol mono(meth)acrylate, N-methylol (meth)acrylamide, N-hydroxyethyl (meth)acrylamide, N,N-(dihydroxyethyl) (meth)acrylamide, methyl (meth)acrylate, ethyl (meth)acrylate, propyl (meth)acrylate, isopropyl (meth)acrylate, butyl (meth)acrylate, isobutyl (meth)acrylate, benzyl (meth)acrylate, lauryl (meth)acrylate, 2,3-dibromopropyl (meth)acrylate, 3-(meth)acryloyloxypropyltrimethoxysilane, 11-(meth)acryloyloxyundecyltrimethoxysilane, or (meth)acrylamide.

Examples of the polymerizable monomer having two radically polymerizable groups include one or more selected from 2,2-bis((meth)acryloyloxyphenyl)propane, 2,2-bis[4-(3-(meth)acryloyloxy)-2-hydroxypropoxyphenyl]propane (commonly known as “Bis-GMA”), 2,2-bis(4-(meth)acryloyloxyphenyl)propane, 2,2-bis(4-(meth)acryloyloxypolyethoxyphenyl)propane, 2,2-bis(4-(meth)acryloyloxydiethoxyphenyl)propane, 2,2-bis(4-(meth)acryloyloxytetraethoxyphenyl)propane, 2,2-bis(4-(meth)acryloyloxypentaethoxyphenyl)propane, 2,2-bis(4-(meth)acryloyloxydipropoxyphenyl)propane, 2-(4-(meth)acryloyloxydiethoxyphenyl)-2-(4-(meth)acryloyloxydiethoxyphenyl)propane, 2-(4-(meth)acryloyloxydiethoxyphenyl)-2-(4-(meth)acryloyloxyditriethoxyphenyl)propane, 2-(4-(meth)acryloyloxydipropoxyphenyl)-2-(4-(meth)acryloyloxytriethoxyphenyl)propane, 2,2-bis(4-(meth)acryloyloxypropoxyphenyl)propane, 2,2-bis(4-(meth)acryloyloxyisopropoxyphenyl)propane, 1,4-bis(2-(meth)acryloyloxyethyl)pyromellitate, glycerol di(meth)acrylate, ethylene glycol di(meth)acrylate, diethylene glycol di(meth)acrylate, triethylene glycol di(meth)acrylate, propylene glycol di(meth)acrylate, butylene glycol di(meth)acrylate, neopentyl glycol di(meth)acrylate, polyethylene glycol di(meth)acrylate, 1,3-butanediol di(meth)acrylate, 1,5-pentanediol di(meth)acrylate, 1,6-hexanediol di(meth)acrylate, 1,10-decanediol di(meth)acrylate, 1,2-bis(3-methacryloyloxy-2-hydroxypropoxy)ethane, 2,2,4-trimethylhexamethylenebis(2-carbamoyloxyethyl)dimethacrylate (commonly known as “UDMA”), or 1,2-bis(3-methacryloyloxy-2-hydroxypropoxy)ethane. Among these, from the viewpoint of improving the mechanical strength of the final composition to be produced from the dental polymerizable composition (β) or the dental polymerizable composition intermediate (γ), preferred are one or more selected from 2,2-bis[4-(3-(meth)acryloyloxy)-2-hydroxypropoxyphenyl]propane, 2,2-bis(4-(meth)acryloyloxypolyethoxyphenyl)propane, or 1,6-bis(methacrylethyloxycarbonylamino)-2,2,4,-trimethylhexane, and from the viewpoint of having an effect of reducing a viscosity of the dental polymerizable composition (β) or the dental polymerizable composition intermediate (γ), preferred are one or more selected from triethylene glycol di(meth)acrylate, neopentyl glycol di(meth)acrylate, or glycerol di(meth)acrylate.

Examples of the polymerizable monomer having three or more radically polymerizable groups include one or more selected from trimethylolpropane tri(meth)acrylate, trimethylolethane tri(meth)acrylate, trimethylolmethane tri(meth)acrylate, pentaerythritol tri(meth)acrylate, pentaerythritol tetra(meth)acrylate, dipentaerythritol penta(meth)acrylate, N,N-(2,2,4-trimethylhexamethylene)bis[2-(aminocarboxy)propane-1,3-diol]tetramethacrylate, or 1,7-diacryloyloxy-2,2,6,6-tetraacryloyloxymethyl-4-oxyheptane. Among these, from the viewpoint of improving the mechanical strength of the final composition to be produced from the dental polymerizable composition (β) or the dental polymerizable composition intermediate (γ), trimethylolpropane tri(meth)acrylate is preferred.

In addition to the above-described polymerizable monomers, an oligomer or prepolymer having at least one or more polymerizable group in the molecule can be used without any limitations. Also, there is no problem even though such an oligomer or a prepolymer may have a substituent such as a fluoro group in the same molecule. The above-described polymerizable monomers can be used singly or in combination with a plurality thereof.

The (a) polymerizable monomer of the present invention may contain (a1) a polymerizable monomer having an acidic group. The (a1) polymerizable monomer having an acidic group that is any polymerizable monomer having one or more polymerizable groups and one or more acidic groups, can be used without limitations. Examples of the acidic group include one or more selected from a phosphoric acid group, a pyrophosphoric acid group, a thiophosphoric acid group, a phosphonic acid group, a sulfonic acid group, or a carboxylic acid group. Containing the (a1) polymerizable monomer having an acidic group enables the final composition to be produced from the dental polymerizable composition (β) or the dental polymerizable composition intermediate (γ) to impart adhesiveness to a tooth structure and a dental prosthetic device.

Examples of the polymerizable monomer having a phosphoric acid group include one or more selected from 2-(meth)acryloyloxyethyl dihydrogen phosphate, 3-(meth)acryloyloxypropyl dihydrogen phosphate, 4-(meth)acryloyloxybutyl dihydrogen phosphate, 5-(meth)acryloyloxypentyl dihydrogen phosphate, 6-(meth)acryloyloxyhexyl dihydrogen phosphate, 7-(meth)acryloyloxyheptyl dihydrogen phosphate, 8-(meth)acryloyloxyoctyl dihydrogen phosphate, 9-(meth)acryloyloxynonyl dihydrogen phosphate, 10-(meth)acryloyloxydecyl dihydrogen phosphate, 11-(meth)acryloyloxyundecyl dihydrogen phosphate, 12-(meth)acryloyloxydodecyl dihydrogen phosphate, 16-(meth)acryloyloxyhexadecyl dihydrogen phosphate, 20-(meth)acryloyloxyicosyl dihydrogen phosphate, bis[2-(meth)acryloyloxyethyl]hydrogen phosphate, bis[4-(meth)acryloyloxybutyl]hydrogen phosphate, bis[6-(meth)acryloyloxyhexyl]hydrogen phosphate, bis[8-(meth)acryloyloxyoctyl]hydrogen phosphate, bis[9-(meth)acryloyloxynonyl]hydrogen phosphate, bis[10-(meth)acryloyloxydecyl]hydrogen phosphate, 1,3-di(meth)acryloyloxypropyl dihydrogen phosphate, 2-(meth)acryloyloxyethylphenyl hydrogen phosphate, 2-(meth)acryloyloxyethyl-2-bromoethyl hydrogen phosphate, or bis[2-[meth]acryloyloxy-(1-hydroxy methyl)ethyl]hydrogen phosphate; acid chlorides, alkali metal salts, or ammonium salts thereof; or (meth)acrylamide compounds in which ester bonds of these compounds have been replaced with amide bonds.

Examples of the polymerizable monomer having a pyrophosphoric acid group include one or more selected from bis[2-(meth)acryloyloxyethyl]pyrophosphate, bis[4-(meth)acryloyloxybutyl]pyrophosphate, bis[6-(meth)acryloyloxyhexyl]pyrophosphate, bis[8-(meth)acryloyloxyoctyl]pyrophosphate, or bis[10-(meth)acryloyloxydecyl]pyrophosphate; acid chlorides, alkali metal salts, or ammonium salts thereof; or (meth)acrylamide compounds in which ester bonds of these compounds have been replaced with amide bonds.

Examples of the polymerizable monomer having a thiophosphoric acid group include one or more selected from 2-(meth)acryloyloxyethyl dihydrogen thiophosphate, 3-(meth)acryloyloxypropyl dihydrogen thiophosphate, 4-(meth)acryloyloxybutyl dihydrogen thiophosphate, 5-(meth)acryloyloxypentyl dihydrogen thiophosphite, 6-(meth)acryloyloxyhexyl dihydrogen thiophosphate, 7-(meth)acryloyloxyheptyl dihydrogen thiophosphite, 8-(meth)acryloyloxyoctyl dihydrogen thiophosphate, 9-(meth)acryloyloxynonyl dihydrogen thiophosphate, 10-(meth)acryloyloxydecyl dihydrogen thiophosphate, 11-(meth)acryloyloxyundecyl dihydrogen thiophosphate, 12-(meth)acryloyloxydodecyl dihydrogen thiophosphate, 16-(meth)acryloyloxyhexadecyl dihydrogen thiophosphate, or 20-(meth)acryloyloxyicosyl dihydrogen thiophosphate; acid chlorides, alkali metal salts, or ammonium salts thereof; or (meth)acrylamide compounds in which ester bonds of these compounds have been replaced with amide bonds.

Examples of the polymerizable monomer having a phosphonic acid group include one or more selected from 2-(meth)acryloyloxyethyl phenyl phosphonate, 5-(meth)acryloyloxypentyl-3-phosphonopropionate, 6-(meth)acryloyloxyhexyl-3-phosphonopropionate, 10-(meth)acryloyloxydecyl-3-phosphonopropionate, 6-(meth)acryloyloxyhexyl-3-phosphonoacetate, or 10-(meth)acryloyloxydecyl-3-phosphonoacetate; acid chlorides, alkali metal salts, or ammonium salts thereof; or (meth)acrylamide compounds in which ester bonds of these compounds have been replaced with amide bonds.

Examples of the polymerizable monomer having a sulfonic acid group include one or more selected from 2-(meth)acrylamide-2-methylpropanesulfonic acid or 2-sulfoethyl (meth)acrylate.

Examples of the polymerizable monomer having a carboxylic acid group include one or more selected from a (meth)acrylic-based compound having one carboxyl group in the molecule, or a (meth)acrylic-based compound having a plurality of carboxyl groups in the molecule. Examples of the (meth)acrylic compound having one carboxyl group in the molecule include one or more selected from (meth)acrylic acid, N-(meth)acryloylglycine, N-(meth)acryloylaspartic acid, 0-(meth)acryloyltyrosine, N-(meth)acryloyltyrosine, N-(meth)acryloylphenylalanine, N-(meth)acryloyl-p-aminobenzoic acid, N-(meth)acryloyl-o-aminobenzoic acid, p-vinylbenzoic acid, 2-(meth)acryloyloxybenzoic acid, 3-(meth)acryloyloxybenzoic acid, 4-(meth)acryloyloxybenzoic acid, N-(meth)acryloyl-5-aminosalicylic acid, N-(meth)acryloyl-4-aminosalicylic acid, 2-(meth)acryloyloxyethyl hydrogen succinate, 2-(meth)acryloyloxyethyl hydrogen phthalate, or 2-(meth)acryloyloxyethyl hydrogen malate; acid halides thereof; or (meth)acrylamide compounds in which ester bonds of these compounds have been replaced with amide bonds. Examples of the (meth)acrylic-based compound having a plurality of carboxyl groups in the molecule include one or more selected from 6-(meth)acryloyloxyhexane-1,1-dicarboxylic acid, 9-(meth)acryloyloxynonane-1,1-dicarboxylic acid, 10-(meth)acryloyloxydecane-1,1-dicarboxylic acid, 11-(meth)acryloyloxyundecane-1,1-dicarboxylic acid, 12-(meth)acryloyloxydodecane-1,1-dicarboxylic acid, 13-(meth)acryloyloxytridecane-1,1-dicarboxylic acid, 4-(meth)acryloyloxyethyl trimellitic acid, 4-(meth)acryloyloxybutyl trimellitic acid, 4-(meth)acryloyloxyhexyl trimellitic acid, 4-(meth)acryloyloxydecyl trimellitic acid, or 2-(meth)acryloyloxyethyl-3′-(meth)acryloyloxy-2′-(3,4-dicarboxybenzoyloxy)propyl succinate; acid anhydrides or acid halides thereof; or (meth)acrylamide compounds in which ester bonds of these compounds have been replaced with amide bonds.

The (a) polymerizable monomer of the present invention may contain (a2) a polymerizable monomer having a sulfur atom. Any publicly known compounds of the (a2) polymerizable monomer having a sulfur atom, can be used without any limitations as long as it is a polymerizable monomer having at least one sulfur atom and one or more polymerizable groups in the molecule. The sulfur atom is contained in a form that does not constitute an acidic group such as a sulfo group in the molecule. The sulfur atom is contained in a form that constitutes a structure other than an acidic group, for example, by forming a structure such as C═S or C—S—C in the molecule. It is to be noted that the (a2) polymerizable monomer having a sulfur atom is preferably free of an acidic group. Examples of the (a2) polymerizable monomer having a sulfur atom include one or more compounds selected from a compound that can generate a mercapto group resulting from tautomerization, a disulfide compound, or a chain or cyclic thioether compound. Examples of the (a2) polymerizable monomer having a sulfur atom include one or more selected from 10-methacryloxydecyl-6,8-dithioctanate, 6-methacryloxyhexyl-6,8-dithioctanate, 6-methacryloyloxyhexyl 2-thiouracil-5-carboxylate, or 2-(11-methacryloyloxyundecylthio)-5-mercapto-1,3,4-thiadiazole. The (a2) polymerizable monomers having a sulfur atom may be used singly or in combination of two or more thereof. Containing the (a2) polymerizable monomer having a sulfur atom enables the final composition to be produced from the dental polymerizable composition (β) or the dental polymerizable composition intermediate (γ) to impart adhesiveness to a noble metal.

### <(b) Polymerization Inhibitor>

The (b) polymerization inhibitor to be compounded in the dental polymerizable composition (B) or the dental polymerizable composition intermediate (γ) of the present disclosure, which is any publicly known polymerization inhibitor, can be used without any limitations. Examples of the polymerization inhibitor include one or more selected from dibutylhydroxytoluene, hydroquinone, dibutylhydroquinone, hydroquinone monomethyl ether, 2,6-t-butylphenol, or 2,6-di-t-butyl-4-methylphenol.

The (b) polymerization inhibitor is preferably compounded in an amount of 5 parts by mass or less relative to 100 parts by mass of the (a) polymerizable monomer. The amount of polymerization inhibitor compounded exceeding 5 parts by mass relative to 100 parts by mass of the (a) polymerizable monomer may possibly prevent the final composition to be produced from the produced dental polymerizable composition (β) or dental polymerizable composition intermediate (γ), from being polymerized and cured.

### <(c) Organic Solvent>

The present invention preferably contains (c) an organic solvent from the viewpoint of improving mixing efficiency. The (c) organic solvent usually has a boiling point of 150° C. or lower under normal pressure and solubility in water of 5% by mass or more at 25° C., more preferably 30% by mass or more. Most preferably, the (c) organic solvent is soluble in water at an arbitrary ratio. Among them, preferred is a water-soluble volatile organic solvent having a boiling point of 100° C. or lower under normal pressure. Examples of the water-soluble volatile organic solvent having a boiling point of 100° C. or lower under normal pressure include one or more selected from ethanol, methanol, 1-propanol, isopropyl alcohol, acetone, methyl ethyl ketone, 1,2-dimethoxyethane, 1,2-diethoxyethane, or tetrahydrofuran. Also, the aforementioned water-soluble volatile organic solvent is preferably one or more selected from acetone, ethanol, or isopropyl alcohol. The (c) organic solvent can be used singly or as a mixture of two or more thereof.

The amount of the (c) organic solvent compounded in the dental polymerizable composition (β) or the dental polymerizable composition intermediate (γ) is preferably selected so that a viscosity of the dental polymerizable composition (β) or the dental polymerizable composition intermediate (γ) to be produced at 25° C. is 5000 mPa-s or less.

### <(d) Polymerization Initiator>

The dental polymerizable composition (β) or the dental polymerizable composition intermediate (γ) may be produced by adding (d) a polymerization initiator to the composition (α) of the present invention.

<(d1) Photopolymerization Initiator>

Examples of the (d) polymerization initiator that can be compounded in the composition (α) of the present invention include (d1) a photopolymerization initiator and (d2) a chemical polymerization initiator. The photopolymerization initiator is a polymerization initiator capable of initiating polymerization by irradiation of light. Examples of the photopolymerization initiator that can be compounded in the composition (α) of the present invention include one or more selected from a photosensitizer, a photoacid generator, or a photopolymerization accelerator. Known compounds commonly used can be used therefor without any limitations. It is to be noted that when the (d1) photopolymerization initiator is compounded in the composition (α), it is preferable to work in a dark room where light in the absorption wavelength range of the (d1) photopolymerization initiator is blocked.

Specific examples of the photosensitizer that can be compounded in the composition (α) of the present invention include one or more selected from α-diketones, benzoin alkyl ethers, thioxanthones, benzophenones, acylphosphine oxides, or an acylgermanium compound. Examples of the α-diketones include one or more selected from camphorquinone, camphorquinone carboxylic acid, or camphorquinone sulfonic acid. Examples of the benzoin alkyl ethers include one or more selected from benzoin, benzoin methyl ether, or benzoin ethyl ether. Examples of the thioxanthones include one or more selected from 2-isopropylthioxanthone, 2-methoxythioxanthone, 2-hydroxythioxanthone, 2,4-diethylthioxanthone, or 2,4-diisopropylthioxanthone. Examples of the benzophenones include one or more selected from benzophenone, p-chlorobenzophenone, or p-methoxybenzophenone. Examples of the acylphosphine oxides include one or more selected from diphenyl(2,4,6-trimethylbenzoyl)phosphine oxide, or phenylbis(2,4,6-trimethylbenzoyl)phosphine oxide. Examples of the acylgermanium compounds include one or more selected from bisbenzoyldiethylgermanium or bisbenzoyldimethylgermanium.

Examples of the photoacid generator that can be compounded in the composition (α) of the present invention include one or more selected from a triazine compound, an iodonium salt-based compound, a sulfonium salt-based compound, or a sulfonic acid ester compound. Among these, one or more compounds selected from a triazine compound or an iodonium salt-based compound are preferred because they have high polymerizability when used in combination with a sensitizer. The iodonium salt-based compound is preferably one or more compounds selected from 4-isopropyl-4′-methyldiphenyliodonium tetrakis(pentafluorophenyl)borate, bis(4-tert-butylphenyl)iodonium tetrakis(pentafluorophenyl)borate, bis(4-tert-butylphenyl)iodonium hexafluorophosphate, diphenyliodonium-2-carboxylate monohydrate, or the like.

The photopolymerization accelerator that can be compounded in the composition (α) of the present invention can be an amine compound. Examples of the amine compound include one or more selected from p-dimethylaminobenzoic acid ethyl ester, triethanolamine, triisopropanolamine, tribenzylamine, dibenzylglycine ethyl ester, N,N-dimethylaminoethyl acrylate, N,N-diethylaminoethyl methacrylate, or N,N-diisopropylaminoethyl methacrylate.

<(d2) Chemical Polymerization Initiator>

An example of the (d) polymerization initiator that can be compounded in the composition (α) of the present invention includes a chemical polymerization initiator. Chemical polymerization is a polymerization method for curing it, without requiring special equipment such as a light irradiator. The chemical polymerization initiator is a polymerization initiator that can initiate chemical polymerization. Known compounds that are commonly used can be used without any limitations.

As specific examples of a transition metal compound that can be compounded in the composition (α) of the present invention, a copper (Cu) compound or a vanadium (V) compound, can be preferably used. Examples of the copper (Cu) compound include one or more compounds selected from copper(I) chloride (monovalent), copper(I) bromide (monovalent), copper(II) chloride (divalent), copper(II) acetate (divalent), copper(II) gluconate (divalent), copper(II) acetylacetonate (divalent), or copper(II) methacrylate (divalent). Examples of the vanadium compound include one or more selected from vanadium acetylacetonate (trivalent), divanadium tetroxide (tetravalent), vanadyl acetylacetonate (tetravalent), vanadium oxide stearate (tetravalent), vanadyl oxalate (tetravalent), vanadyl sulfate (tetravalent), oxo-bis(1-phenyl-1,3-butanedionate) vanadium (tetravalent), bis(maltolato)oxovanadium (tetravalent), vanadium pentoxide (pentavalent), or sodium metavanadate (pentavalent).

Examples of a thiourea compound that can be compounded in the composition (α) of the present invention include one or more selected from dimethylthiourea, diethylthiourea, tetramethylthiourea, (2-pyridyl)thiourea, N-methylthiourea, ethylenethiourea, N-allylthiourea, N-allyl-N′-(2-hydroxyethyl)thiourea, N-benzylthiourea, 1,3-dicyclohexylthiourea, N,N′-diphenylthiourea, 1,3-di(p-tolyl)thiourea, 1-methyl-3-phenylthiourea, N-acetylthiourea, N-benzoylthiourea, diphenylthiourea, or dicyclohexylthiourea. Among these, preferred are one or more selected from (2-pyridyl)thiourea, N-acetylthiourea, N-benzoylthiourea, or N-benzylthiourea.

Examples of an organic peroxide that can be compounded in the composition (α) of the present invention include one or more selected from diacyl peroxides, peroxy esters, dialkyl peroxides, peroxy ketals, ketone peroxides, peroxy esters, peroxy dicarbonates, or hydroperoxides. Among these, examples thereof include one or more selected from t-butylperoxy-2-ethylhexanoate, t-butylperoxybenzoate, t-amylperoxy-2-ethylhexanoate, t-amylperoxyacetate, t-amylperoxybenzoate, 1,1-di(t-butylperoxy)cyclohexane, 1,1-di(t-amylperoxy)cyclohexane, dibenzoyl peroxide, cumene hydroperoxide, t-butyl hydroperoxide, or 1,1,3,3-tetramethylbutyl hydroperoxide.

Examples of other chemical polymerization initiator include one or more selected from a phosphine compound, a sulfinic acid compound, a borate compound, a barbituric acid derivative, or an ascorbic acid compound. Examples of the phosphine compound include one or more selected from triphenylphosphine or 4-(phenylphosphino)benzoic acid. Examples of the sulfinic acid compound include one or more selected from sodium benzenesulfinate, sodium p-toluenesulfinate, or sodium 2,4,6-triisopropylbenzenesulfinate. Examples of the borate compound include one or more selected from salts (one or more selected from a sodium salt, a lithium salt, a potassium salt, or the like) of tetraarylborate compounds or a tetrabutylammonium salt. Examples of the barbituric acid derivative include one or more selected from 5-butylbarbituric acid, 1,3,5-trimethylbarbituric acid, 1-cyclohexyl-5-ethylbarbituric acid, or salt (a sodium salt or a calcium salt) of the barbituric acid derivative described above. Examples of the ascorbic acid compound include one or more selected from ascorbic acid, ascorbyl 6-palmitate, or a salt compound of the above-described ascorbic acid compound.

### <(e) Filler>

The (e) filler has a possibility to contribute to improving the mechanical strength of the dental polymerizable composition (β) or the dental polymerizable composition intermediate (γ). However, it is difficult to homogeneously disperse the (e) filler in the composition (α). This is because a stirrer has a structure in which a production machine is equipped with a container but is substantially not equipped with a stirring blade, and the container operates in one or more modes selected from rotation, hyperbolic motion, or vibration. Therefore, it is preferable not to substantially compound the (e) filler in the dental polymerizable composition (β) or the dental polymerizable composition intermediate (γ) of the present invention. With the proviso that the step of dispersing the (e) filler in the dental polymerizable composition intermediate (γ), produced in the present invention may be included in the subsequent production step. An example of the method for dispersing the (e) filler in the dental polymerizable composition intermediate (γ) include a method for kneading it using a production machine such as a planetary centrifugal mixer, a dissolver, or a planetary mixer, and removing air bubbles under reduced pressure, if necessary. In particular, the dispersion method using a planetary centrifugal mixer is preferable because it does not include a stirring blade, and thereby can obtain the final mixture in high yield. The phrase “substantially free of filler” as used herein means that the composition (α) is free of the (e) filler.

Examples of the (e) filler includes (e1) an inorganic filler, (e2) an organic filler, and (e3) an organic-inorganic composite filler.

Examples of the (e1) inorganic filler include one or more selected from silica, a mineral containing silica as a base material, a ceramic containing silica as a base material and a metal oxide, or glass. Examples of the mineral containing silica as a base material include one or more selected from kaolin, clay, mica, or mica. Examples of the metal oxide include one or more selected from Al₂O₃, B₂O₃, TiO₂, ZrO₂, BaO, La₂O₃, SrO₂, CaO, or P₂O₅. Examples of the glasses include one or more selected from lanthanum glass, barium glass, strontium glass, soda glass, lithium borosilicate glass, zinc glass, fluoroaluminosilicate glass, borosilicate glass, or bioglass, which have been suitably used in the dental field. Examples of the (e1) inorganic filler include one or more selected from crystalline quartz, hydroxyapatite, alumina, titanium oxide, yttrium oxide, zirconia, calcium phosphate, barium sulfate, aluminum hydroxide, sodium fluoride, potassium fluoride, sodium monofluorophosphate, lithium fluoride, or ytterbium fluoride.

Examples of the (e2) organic filler include one or more selected from polymethyl methacrylate, polyethyl methacrylate, a polyfunctional methacrylate polymer, polyamide, polystyrene, polyvinyl chloride, chloroprene rubber, nitrile rubber, or styrene-butadiene rubber.

The (e3) organic-inorganic composite filler is a filler containing an inorganic filler and a polymer of monomers, and is obtained by preliminarily adding a polymerizable monomer to an inorganic filler, forming into paste, polymerizing it, and then pulverizing the polymer.

An example of the (e) filler also includes a filler that has been preliminarily surface-treated with a publicly known surface treatment agent such as a silane coupling agent. Examples of the surface treatment agent include one or more selected from vinyltrimethoxysilane, vinyltriethoxysilane, vinyltrichlorosilane, vinyltri(0-methoxyethoxy)silane, γ-methacryloyloxypropyltrimethoxysilane, γ-glycidoxypropyltrimethoxysilane, γ-mercaptopropyltrimethoxysilane, or γ-aminopropyltriethoxysilane.

An example of the (e) filler also includes a fine particle filler having a primary particle size of 0.001 to 0.1 μm. Specific examples thereof include “Aerosil OX50,” “Aerosil 50,” “Aerosil 200,” “Aerosil 380,” “Aerosil R972,” “Aerosil R974,” “Aerosil 130,” “Aerosil RX200,” or “Aerosil R711” (all of which are product names, manufactured by Evonik Industries AG).

### <Other Components>

As long as the effect of the present invention is not impaired, one or more components selected from water, an ultraviolet-ray absorber, an α-alkylstyrene compound, a mercaptan compound, a chain transfer material, a metal scavenger, a discoloration inhibitor, an antibacterial material, other conventionally publicly known additives, or the like may be added to the composition (α) of the present invention, if necessary to produce the dental polymerizable composition (β) or the dental polymerizable composition intermediate (γ). Examples of the ultraviolet-ray absorber include one or more selected from benzophenone-based or benzotriazole-based ultraviolet-ray absorber. Examples of the mercaptan compound include one or more selected from n-butyl mercaptan or n-octyl mercaptan. An example of the chain transfer agent includes a terpenoid-based compound. Examples of the terpenoid-based compound include one or more selected from limonene, myrcene, α-terpinene, β-terpinene, γ-terpinene, terpinolene, β-pinene, or α-pinene. Examples of the metal scavenger include one or more selected from an aminocarboxylic acid-based chelating agent or a phosphonic acid-based chelating agent.

<Production Machine Equipped with Container>

The production machine equipped with a container in the present invention can be used without any particular limitations as long as it is a machine that is not substantially equipped with a stirring blade, has a container into which the composition (α) can be charged, and can mix components in the container. The production machine equipped with a container is preferably (1) a stirrer whereby a container rotates on its axis, (2) a stirrer whereby a container moves in hyperbolic motion, or (3) a stirrer whereby a container vibrates. Using the (1) stirrer whereby a container rotates on its axis, the (2) stirrer whereby a container moves in hyperbolic motion, or the (3) stirrer whereby a container vibrates, enables facilitating the dental polymerizable composition (β) or the dental polymerizable composition intermediate (γ), which is substantially free of a filler and has excellent storage stability to be obtained in high yield. The rotation refers to movement of a container itself around one point or axis of the container. The hyperbolic motion refers to movement of a container so as to draw the shape of a hyperbola. Specifically, it means that the motion whereby a container moves in one direction, then moves in the opposite direction, and then moves again in the original direction, is repeated as a cycle. The hyperbolic motion may allow the container not only to move horizontally, but also to move at an inclined angle. The vibration refers to a repeated reciprocating motion of the container back and forth, up and down, left and right, or the like.

The (1) stirrer whereby a container rotates on its axis may also be a commercially available stirrer. Examples of the (1) stirrer whereby a container rotates on its axis include the product name “Tabletop Tumbler Lab” (EiShin Co., Ltd.), the product name “Mobile Tumbler Lab” (EiShin Co., Ltd.), the product name “Tumbler Mini” (EiShin Co., Ltd.), the product name “Tumbler Mixer” (EiShin Co., Ltd.), the product name “Drum Mixer” (EiShin Co., Ltd.), or the product name “SK-1100TVII” (Shashin Kagaku Co., Ltd.).

In case of using the (1) stirrer whereby a container rotates on its axis, the rotation speed (min⁻¹) and operation time are not particularly limited as long as the composition (α) is stirred and mixed to produce a dental polymerizable composition (β) or the dental polymerizable composition intermediate (γ), which is substantially free of a filler, however, there is a possibility of generation of heat of mixing or a decrease in mutual solubility of the raw materials, so that the rotation speed is preferably 100 min⁻¹ or less and more preferably 60 min⁻¹ or less.

The (2) stirrer whereby a container moves in hyperbolic motion may be a commercially available stirrer. Examples of the (2) stirrer whereby a container moves in hyperbolic motion include the product name “Turbla Shaker Mixer T2G” (Shinmaru Enterprise Corporation), the product name “Turbla Shaker Mixer T2GE” (Shinmaru Enterprise Corporation), the product name “Turbla Shaker Mixer T2F” (Shinmaru Enterprise Corporation), the product name “Turbla Shaker Mixer T10B” (Shinmaru Enterprise Corporation), or the product name “Turbla Shaker Mixer T50A” (Shinmaru Enterprise Corporation).

In case of using the (2) stirrer whereby a container moves in hyperbolic motion, the speed (min⁻¹) of the hyperbolic motion and operation time are not particularly limited as long as the composition (α) is stirred and mixed to produce a dental polymerizable composition (β) or the dental polymerizable composition intermediate (γ), which is substantially free of a filler, however, there is a possibility of heat of mixing being generated, so that the speed of the hyperbolic motion is preferably 50 min⁻¹ or less.

The (3) stirrer whereby a container vibrates may be a commercially available stirrer. Examples of the (3) stirrer whereby a container vibrates include the product name “VORTEX 3” (IKA Japan K. K.), the product name “VORTEX Genius 3” (IKA Japan K. K.), the product name “CPS-20” (Biosan SIA), the product name “Rocking shaker” (Seiwa Giken Co., Ltd.), the product name “Vacuum defoaming Vibratory stirrer VVS-10” (Seiwa Giken Co., Ltd.), and the product name “Vacuum defoaming Vibratory stirrer VS-15” (Seiwa Giken Co., Ltd.).

In case of using the (3) stirrer whereby a container vibrates, the vibration speed (min⁻¹) and operation time are not particularly limited as long as the composition (α) is stirred and mixed to produce a dental polymerizable composition (β) or the dental polymerizable composition intermediate (γ), which is substantially free of a filler, however, there is a possibility of heat of mixing being generated, so that the vibration speed is preferably 400 min⁻¹ or less.

The container in the production machine of the present invention is made of a material that does not react with the component constituting the composition (α) and can be used without any particular limitations as long as it has a shape that can be equipped in the production machine. The container is preferably charged with the composition (α) such that an empty space rate of the container is 10 to 90% in consideration of its mixing efficiency, and is more preferably charged with the composition (α) such that an empty space rate of the container is 20 to 80%. The empty space rate of the container being less than 10% or more than 90% reduces the mixing efficiency of the composition (α), making it difficult to obtain a homogeneous composition (α).

One example of the production method disclosed in the present invention is a production method including the steps of:
1-1: disposing a composition (α) containing (a) a polymerizable monomer and (b) a polymerization inhibitor in a container of a production machine that is substantially not equipped with a stirring blade and is equipped with a container such that an empty space rate is 10 to 90%; and
1-2: allowing the container to operate.

An example of the production method disclosed in the present invention is a production method including the steps of:
2-1: disposing a composition (α) containing (a) a polymerizable monomer and (b) a polymerization inhibitor in a container of a production machine that is substantially not equipped with a stirring blade and is equipped with a container such that an empty space rate is 10 to 90%;
2-2: heating the container to 30 to 70° C.; and
2-3: allowing the container to operate.

The phrase “substantially not equipped with a stirring blade” as used herein means that the production machine is not equipped with a stirring portion for stirring, that is, a blade or vane attached to a rotating shaft of the apparatus. The phrase “empty space rate” as used herein means ([space that does not contain various substances (polymerizable monomer having flowability and/or an organic solvent and/or water, and the like,)]/[total space of the container])×100. The 1-1 and 2-1 steps are preferably carried out at room temperature (approximately 10 to 30° C.). The phrase “allowing the container to operate” as used herein means that the container operates in one or more modes selected from rotation, hyperbolic motion, or vibration. In case of carrying out the “step of heating the container to between 3° and 70° C.,” the viscosity of the (a) polymerizable monomer decreases and the mixing efficiency of the composition (α) is improved, so that this step is preferably carried out.

The following step (L) before 1-1 or 2-1 is considered to be included. It is possible to obtain (a) a homogeneous polymerizable monomer through the step (L). Carrying out the following step (L) before 1-1 or 2-1 allows the raw materials to be homogeneously mixed to shorten the time required for the dental polymerizable composition (β) or the dental polymerizable composition intermediate (γ) to be obtained.
(L) stirring multiple types of (a) polymerizable monomers

In addition to 1-1 to 1-2 and 2-1 to 2-3, the following steps (M) to (0) are considered to be included. These steps may be carried out either before or after the steps 1-1 to 1-2, and the steps 2-1 to 2-3. In case of carrying out the following steps (M) to (O) after the “step of allowing the container to operate” in the production method of the present invention, a product to be obtained is considered to be homogenized by stirring it using the production machine that is not substantially equipped with a stirring blade but is equipped with a container, or a production machine that is equipped with a stirring blade and a container. The following steps (M) to (O) are preferably carried out after the 1-1 to 1-2 and 2-1 to 2-3 steps.
(M) disposing (c) an organic solvent in a container of a production machine that is substantially not equipped with a stirring blade and is equipped with a container.
(N) disposing (d) a polymerization initiator in a container of a production machine that is substantially not equipped with a stirring blade and is equipped with a container.
(O) disposing other components in a container of a production machine that is substantially not equipped with a stirring blade and is equipped with a container.

The step (P) may be carried out following the production method disclosed in the present invention. Also, in the step (P), a method for repeating the step of disposing the (e) filler in a container followed by stirring it to gradually disperse the (e) filler, is considered to be employed.
(P) disposing the dental polymerizable composition (β) or the dental polymerizable composition intermediate (γ) and the (e) filler in a container of a production machine that is substantially equipped with a stirring blade and the container, and stirring them.

### EXAMPLES

The materials and their abbreviations used in Examples and Comparative Examples are shown below. It is to be noted that the present invention is not limited to these Examples.

### <(a) Polymerizable Monomer>

BisGMA: 2,2-Bis[4-(3-methacryloyloxy-2-hydroxypropoxy)phenyl]propane (density: approximately 1.16 g/mL at 25° C.)
UDMA: N,N-(2,2,4-trimethylhexamethylene)bis[2-(aminocarboxy)ethanol]methacrylate (density: approximately 1.11 g/mL at 25° C.)
TEGDMA: Triethylene glycol dimethacrylate (density: approximately 1.07 g/mL at 25° C.)
GDMA: Glycerol dimethacrylate (density: approximately 1.12 g/mL at 25° C.)
HEMA: Hydroxyethyl methacrylate (density: approximately 1.02 g/mL at 25° C.)
MDP: 10-Methacryloyloxydecyl dihydrogen phosphate (density: approximately 1.14 g/mL at 25° C.)
MHPA: 6-Methacryloxyhexyl phosphonoacetate (density: approximately 1.20 g/mL at 25° C.)
META: 4-Methacryloyloxyethoxycarbonyl phthalic anhydride (the density (g/mL at 25° C.) is not listed because it is powder).

### <(b) Polymerization Inhibitor>

MEHQ: Hydroquinone monomethyl ether
BHT: Dibutyl hydroxytoluene

### <(c) Organic Solvent>

Acetone (density: approximately 0.78 g/mL at 25° C.)
Ethanol (density: approximately 0.78 g/mL at 25° C.)

### <(d) Polymerization Initiator>

CQ: Camphorquinone
BAPO: Phenylbis(2,4,6-trimethylbenzoyl)phosphine oxide
DMBE: 4-N,N-Dimethylaminobenzoic acid ethyl ester
IPIFP: 4-Isopropylphenyl(p-tolyl)iodonium tris(pentafluoroethyl) trifluorophosphate
DEPT: N,N-Di(2-hydroxyethyl)-p-toluidine
BPO: Dibenzoyl peroxide
CHP: Cumene hydroperoxide (density: approximately 1.02 g/mL at 25° C.)
TMBH: 1,1,3,3,-Tetramethylbutyl hydroperoxide (density: approximately 0.89 g/mL at 25° C.)
VOA: Vanadyl acetylacetonate
COA: Copper acetylacetonate
PTU: N-Pyridylthiourea

### <(e) Filler>

R-974: Aerosil R-974 (primary particle size 12 nm, hydrophobized silica surface-treated with dimethyldichlorosilane, manufactured by Evonik Industries AG)
   <Production Machine Equipped with Container>
Production machine 1: Tabletop Tumbler Lab (Eishin Co., Ltd.)
Production machine 2: Turbla Shaker Mixer T2G (Shinmaru Enterprises Co., Ltd.)
Production machine 3: Rocking shaker (Seiwa Giken Co., Ltd.)
   <Production Machine Other than Machine Equipped with Container>
Production machine 4: Mazera ZZ-1200 (TOKYO RIKAKIKAI CO., LTD.) with a stirring blade: T-1A (TOKYO RIKAKIKAI CO., LTD.)

### Example 1

A transparent polyethylene container with a volume of 2 L was charged with 138 g of BisGMA, 92 g of GDMA, 0.23 g of BHT, 2.3 g of CQ, and 2.3 g of DMBE. The container was closed with a lid and heated in a thermostatic chamber at 50° C. for 1 hour, after which the container was installed in a production machine 2, and the contents were mixed at a speed of 50 min⁻¹ to prepare a mixture.

### Example 2

A transparent polyethylene container with a volume of 2 L was charged with 1,212 g of BisGMA, 808 g of GDMA, 2.02 g of MEHQ, 20.2 g of CQ, 20.2 g of DMBE, and 20.2 g of DEPT. The container was closed with a lid and heated in a thermostatic chamber at 50° C. for 1 hour, after which the container was installed in a production machine 1, and the contents were mixed at a speed of 60 min⁻¹ to prepare a mixture.

### Example 3

A transparent polyethylene container with a volume of 2 L was charged with 132 g of UDMA, 88 g of TEGDMA, and 2.2 g of BHT. The container was closed with a lid, the container was installed in a production machine 3, and the contents were mixed at a speed of 400 min⁻¹ to prepare a mixture.

### Examples 4 to 27 and Comparative Examples 1 to 13

Each mixture was prepared by changing the component to be compounded and the amount thereof without changing the mixing conditions described in any one of Examples 1 to 3 (in case of using the same machine, the same speed was employed). In the case of the composition that was free of an organic solvent but contained BisGMA, the container was charged with all components, which were then heated in a thermostatic chamber at 50° C. for 1 hour followed by carrying out the mixing step.

### Example 28

A transparent polyethylene container with a volume of 2 L was charged with 138 g of BisGMA, 92 g of GDMA, 0.23 g of BHT, and 2.3 g of CQ. The container was closed with a lid and heated in a thermostatic chamber at 50° C. for 1 hour, after which the container was installed in a production machine 2, and the contents were mixed at a speed of 50 min⁻¹. Five hours after the start of mixing, the container was temporarily removed from the production machine and then added with 2.3 g of CHP. Thereafter, the container was again installed in production machine 2 and the contents were mixed at a speed of 50 min⁻¹ to prepare a mixture.

### Example 29

A transparent polyethylene container with a volume of 2 L was charged with 1,212 g of BisGMA, 808 g of GDMA, 2.02 g of MEHQ, 20.2 g of CQ, and 20.2 g of DMBE. The container was closed with a lid and heated in a thermostatic chamber at 50° C. for 1 hour, after which the container was installed in a production machine 1, and the contents were mixed at a speed of 60 min⁻¹. Five hours after the start of mixing, the container was temporarily removed from the production machine and then added with 20.2 g of TMBH. Thereafter, the container was again installed in production machine 1 and the contents were mixed at a speed of 60 min⁻¹ to prepare a mixture.

### Example 30

A transparent polyethylene container with a volume of 2 L was charged with 132 g of UDMA, 88 g of TEGDMA, 0.22 g of BHT, and 2.2 g of CQ. The container was closed with a lid, the container was installed in a production machine 3, and the contents were mixed at a speed of 400 min⁻¹. Five hours after the start of mixing, the container was temporarily removed from the production machine and then added with 2.2 g of BPO. Thereafter, the container was again installed in production machine 3, and the contents were mixed at a speed of 400 min⁻¹ to prepare a mixture.

### Examples 31 to 33 and Comparative Examples 14 and 15

Each mixture was prepared by changing the component to be compounded and the amount thereof without changing the mixing conditions described in any one of Examples 28 to 30 (in case of using the same machine, the same speed was employed). In the case of the composition that contained BisGMA, the container was charged with the components other than the organic peroxide, after which the contents were heated in a thermostatic chamber at 50° C. for 1 hour and then mixed for 5 hours. Thereafter, the container was temporarily removed from the production machine and added with the organic peroxide, and the contents were mixed again to prepare a mixture.

### Comparative Example 16

A transparent polyethylene container with a volume of 2 L was charged with 672 g of BisGMA, 448 g of TEGDMA, 1.12 g of MEHQ, 11.2 g of CQ, 11.2 g of DMBE, and 11.2 g of DEPT. The stirring blade attached to a production machine 4 was installed into the container, and the contents were mixed at a speed of 600 min⁻¹ to prepare a mixture.

### Comparative Examples 17 to 21

Each mixture was prepared by changing the components to be compounded without changing the mixing conditions described in Comparative Example 16 (in case of using the same machine, the same speed was employed).

### {Mixing Time Required to Reach Homogeneity}

A time required from the start of the mixing step until the compounded components were homogeneously dissolved was evaluated as the “mixing time required to reach homogeneity.” Whether or not the compounded components were homogeneously dissolved was visually determined. In is to be noted that in a case in which the mixing step was divided into multiple stages, the total time required for each mixing step, was taken as the “mixing time required to reach homogeneity.”

### {Yield}

The weight of the container immediately after charge of raw materials (m1 weight), is measured. The weight of the container that was removed from the stirrer after completion of the mixing step (m2 weight) is measured. The value obtained by dividing the m2 weight by the ml weight and multiplying the result by 100, which was less than 99.9%, is considered “poor”, and the value of 99.9% or more is considered “favorable”.

### {Viscosity Measurement Method}

The mixture obtained was fed in a 50 mL glass bottle and left to stand for one day in a thermostatic chamber at 25° C. After having left it to stand for one day, the viscosity (mPa·S) was measured at 25° C. using a B-type viscometer (BMII type viscometer, manufactured by Toki Sangyo Co., Ltd.). It is to be noted that the viscosity was determined as the value obtained 3 minutes after the start of the measurement.

### {Storage Stability}

The mixture obtained was fed in a 50 mL glass bottle and stored in a thermostatic chamber set at 50° C. for 10 weeks. The mixture was evaluated for changes in its “appearance” and “viscosity” before and after storage at 50° C. When a change was observed, it was marked “X,” and when no change was observed, it was marked “O.” It is to be noted that the appearance of the mixture was visually evaluated. Also, the viscosity was evaluated using the method described above.

TABLE 1

| | | Example 1 | Example 2 | Example 3 | Example 4 | Example 5 | Example 6 |
|---|---|---|---|---|---|---|---|
| (a) | BisGMA | 138 | 1212 | 0 | 0 | 270 | 0 |
| | UDMA | 0 | 0 | 132 | 262.8 | 0 | 1050 |
| | TEGDMA | 0 | 0 | 88 | 0 | 0 | 700 |
| | GDMA | 92 | 808 | 0 | 175.2 | 180 | 0 |
| | HEMA | 0 | 0 | 0 | 0 | 0 | 0 |
| | MDP | 0 | 0 | 0 | 0 | 0 | 0 |
| | MHPA | 0 | 0 | 0 | 0 | 0 | 0 |
| | META | 0 | 0 | 0 | 0 | 0 | 0 |
| (b) | MEHQ | 0 | 2.02 | 0 | 0 | 0.45 | 0 |
| | BHT | 0.23 | 0 | 2.2 | 0.438 | 0 | 1.75 |
| (c) | Acetone | 0 | 0 | 0 | 0 | 0 | 0 |
| | Ethanol | 0 | 0 | 0 | 0 | 0 | 0 |
| (d) | CQ | 2.3 | 20.2 | 0 | 4.38 | 4.5 | 17.5 |
| | BAPO | 0 | 0 | 0 | 0 | 4.5 | 0 |
| | DMBE | 2.3 | 20.2 | 0 | 4.38 | 4.5 | 17.5 |
| | IPIFP | 0 | 0 | 0 | 0 | 0 | 17.5 |
| | DEPT | 0 | 20.2 | 0 | 4.38 | 0 | 0 |
| | BPO | 0 | 0 | 0 | 0 | 0 | 0 |
| | CHP | 0 | 0 | 0 | 0 | 0 | 0 |
| | TMBH | 0 | 0 | 0 | 0 | 0 | 0 |
| | VOA | 0 | 0 | 0 | 0 | 0 | 0 |
| | COA | 0 | 0 | 0 | 0 | 0 | 0 |
| | PTU | 0 | 0 | 0 | 0 | 0 | 0 |
| (e) | R-974 | 0 | 0 | 0 | 0 | 0 | 0 |

| | | | | | | |
|---|---|---|---|---|---|---|
| Empty space rate (%) | 90 | 10 | 90 | 80 | 80 | 20 |
| Production machine 1 | | ∘ | | | ∘ | |
| Production machine 2 | ∘ | | | ∘ | | |
| Production machine 3 | | | ∘ | | | ∘ |
| Production machine 4 | | | | | | |
| Mixing time required | 14 | 14 | 12 | 8 | 12 | 9 |
| to reach homogeneity | | | | | | |
| Viscosity of the final | 849 | 853 | 151 | 162 | 851 | 153 |
| product mPa · s | | | | | | |
| Yield | ∘ | ∘ | ∘ | ∘ | ∘ | ∘ |
| Storage stability | ∘ | ∘ | ∘ | ∘ | ∘ | ∘ |

| | | Example 7 | Example 8 | Example 9 | Example 10 | Example 11 |
|---|---|---|---|---|---|---|
| (a) | BisGMA | 138 | 1260 | 0 | 0 | 285.6 |
| | UDMA | 0 | 0 | 132 | 264 | 0 |
| | TEGDMA | 0 | 0 | 77 | 0 | 0 |
| | GDMA | 80.5 | 735 | 0 | 154 | 166.6 |
| | HEMA | 0 | 21 | 0 | 0 | 0 |
| | MDP | 11.5 | 0 | 11 | 22 | 0 |
| | MHPA | 0 | 0 | 0 | 0 | 0 |
| | META | 0 | 84 | 0 | 0 | 23.8 |
| (b) | MEHQ | 0 | 2.1 | 0 | 0.44 | 0.476 |
| | BHT | 0.23 | 0 | 0.22 | 0 | 0 |
| (c) | Acetone | 0 | 0 | 0 | 0 | 0 |
| | Ethanol | 0 | 0 | 0 | 0 | 0 |
| (d) | CQ | 2.3 | 0 | 2.2 | 0 | 0 |
| | BAPO | 2.3 | 21 | 0 | 0 | 4.76 |
| | DMBE | 0 | 0 | 0 | 4.4 | 0 |
| | IPIFP | 0 | 0 | 0 | 0 | 4.76 |
| | DEPT | 0 | 0 | 0 | 0 | 0 |
| | BPO | 0 | 0 | 0 | 0 | 0 |
| | CHP | 0 | 0 | 0 | 0 | 0 |
| | TMBH | 0 | 0 | 0 | 0 | 0 |
| | VOA | 2.3 | 0 | 2.2 | 0 | 0 |
| | COA | 2.3 | 0 | 0 | 0 | 0 |
| | PTU | 0 | 21 | 2.2 | 0 | 0 |
| (e) | R-974 | 0 | 0 | 0 | 0 | 0 |

| | | | | | |
|---|---|---|---|---|---|
| Empty space rate (%) | 90 | 10 | 90 | 80 | 80 |
| Production machine 1 | | ∘ | | | ∘ |
| Production machine 2 | ∘ | | | ∘ | |
| Production machine 3 | | | ∘ | | |
| Production machine 4 | | | | | |
| Mixing time required | 14 | 14 | 12 | 8 | 12 |
| to reach homogeneity | | | | | |
| Viscosity of the final | 848 | 904 | 149 | 165 | 903 |
| product mPa · s | | | | | |
| Yield | ∘ | ∘ | ∘ | ∘ | ∘ |
| Storage stability | ∘ | ∘ | ∘ | ∘ | ∘ |

TABLE 2

| | | Example 12 | Example 13 | Example 14 | Example 15 | Example 16 | Example 17 |
|---|---|---|---|---|---|---|---|
| (a) | BisGMA | 0 | 0 | 0 | 156 | 282 | 0 |
| | UDMA | 1050 | 651 | 200 | 0 | 0 | 1472 |
| | TEGDMA | 612.5 | 0 | 0 | 0 | 117.5 | 0 |
| | GDMA | 0 | 0 | 0 | 0 | 0 | 92 |
| | HEMA | 0 | 651 | 12.5 | 52 | 0 | 184 |
| | MDP | 87.5 | 0 | 0 | 26 | 0 | 0 |
| | MHPA | 0 | 651 | 12.5 | 0 | 70.5 | 0 |
| | META | 0 | 217 | 25 | 26 | 0 | 92 |
| (b) | MEHQ | 0 | 108.5 | 0 | 5.2 | 0 | 92 |
| | BHT | 1.75 | 0 | 7.5 | 0 | 9.4 | 0 |
| (c) | Acetone | 0 | 0 | 0 | 0 | 0 | 0 |
| | Ethanol | 0 | 0 | 0 | 0 | 0 | 0 |
| (d) | CQ | 0 | 21.7 | 2.5 | 2.6 | 4.7 | 18.4 |
| | BAPO | 0 | 0 | 0 | 2.6 | 0 | 0 |
| | DMBE | 0 | 21.7 | 2.5 | 2.6 | 4.7 | 18.4 |
| | IPIFP | 0 | 0 | 0 | 0 | 0 | 0 |
| | DEPT | 0 | 21.7 | 0 | 0 | 0 | 0 |
| | BPO | 0 | 0 | 0 | 0 | 0 | 0 |
| | CHP | 0 | 0 | 0 | 0 | 0 | 0 |
| | TMBH | 0 | 0 | 0 | 0 | 0 | 0 |
| | VOA | 0 | 0 | 0 | 0 | 0 | 0 |
| | COA | 0 | 0 | 0 | 0 | 0 | 0 |
| | PTU | 0 | 0 | 0 | 0 | 0 | 0 |
| (e) | R-974 | 0 | 0 | 0 | 0 | 0 | 0 |

| Empty space rate (%) | | 20 | 10 | 90 | 90 | 80 | 20 |
|---|---|---|---|---|---|---|---|
| Production machine 1 | | | | | ∘ | | |
| Production machine 2 | | | ∘ | | | ∘ | |
| Production machine 3 | | ∘ | | ∘ | | | ∘ |
| Production machine 4 | | | | | | | |
| Mixing time required | | 9 | 26 | 28 | 29 | 23 | 23 |
| to reach homogeneity | | | | | | | |
| Viscosity of the final | | 155 | 4320 | 7980 | 7990 | 8000 | 6980 |
| product mPa · s | | | | | | | |
| Yield | | ∘ | ∘ | ∘ | ∘ | ∘ | ∘ |
| Storage stability | | ∘ | ∘ | ∘ | ∘ | ∘ | ∘ |
| | | | Example 18 | Example 19 | Example 20 | Example 21 | Example 22 |
| | (a) | BisGMA | 0 | 60 | 0 | 0 | 462 |
| | | UDMA | 405 | 0 | 410.8 | 67.2 | 0 |
| | | TEGDMA | 22.5 | 10 | 94.8 | 0 | 77 |
| | | GDMA | 22.5 | 0 | 0 | 9.6 | 0 |
| | | HEMA | 0 | 10 | 63.2 | 9.6 | 77 |
| | | MDP | 0 | 10 | 0 | 0 | 77 |
| | | MHPA | 0 | 0 | 63.2 | 0 | 0 |
| | | META | 0 | 10 | 0 | 9.6 | 77 |
| | (b) | MEHQ | 0 | 0 | 0 | 0 | 0 |
| | | BHT | 9 | 0.5 | 0.632 | 0.96 | 3.85 |
| | (c) | Acetone | 0 | 100 | 0 | 0 | 770 |
| | | Ethanol | 0 | 0 | 948 | 96 | 0 |
| | (d) | CQ | 4.5 | 1 | 6.32 | 0.96 | 7.7 |
| | | BAPO | 0 | 0 | 0 | 0.96 | 0 |
| | | DMBE | 4.5 | 1 | 6.32 | 0.96 | 7.7 |
| | | IPIFP | 0 | 0 | 0 | 0 | 0 |
| | | DEPT | 0 | 1 | 0 | 0 | 7.7 |
| | | BPO | 0 | 0 | 0 | 0 | 0 |
| | | CHP | 0 | 0 | 0 | 0 | 0 |
| | | TMBH | 0 | 0 | 0 | 0 | 0 |
| | | VOA | 0 | 0 | 0 | 0 | 0 |
| | | COA | 0 | 0 | 0 | 0 | 0 |
| | | PTU | 0 | 0 | 0 | 0 | 0 |
| | (e) | R-974 | 0 | 0 | 0 | 0 | 0 |

| | | | | | |
|---|---|---|---|---|---|
| Empty space rate (%) | 80 | 90 | 10 | 90 | 20 |
| Production machine 1 | ∘ | | | ∘ | |
| Production machine 2 | | ∘ | | | ∘ |
| Production machine 3 | | | ∘ | | |
| Production machine 4 | | | | | |
| Mixing time required | 24 | 4 | 3 | 4 | 2 |
| to reach homogeneity | | | | | |
| Viscosity of the final | 7450 | 31 | 4 | 7 | 29 |
| product mPa · s | | | | | |
| Yield | ∘ | ∘ | ∘ | ∘ | ∘ |
| Storage stability | ∘ | ∘ | ∘ | ∘ | ∘ |

TABLE 3

| | | Example 23 | Example 24 | Example 25 | Example 26 | Example 27 | Example 28 |
|---|---|---|---|---|---|---|---|
| (a) | BisGMA | 0 | 0 | 0 | 0 | 0 | 138 |
| | UDMA | 114 | 343 | 0 | 0 | 0 | 0 |
| | TEGDMA | 0 | 49 | 0 | 0 | 0 | 0 |
| | GDMA | 19 | 0 | 0 | 0 | 0 | 92 |
| | HEMA | 19 | 49 | 0 | 0 | 0 | 0 |
| | MDP | 0 | 0 | 12 | 0 | 12 | 0 |
| | MHPA | 19 | 0 | 0 | 94.4 | 0 | 0 |
| | META | 19 | 49 | 3 | 23.6 | 3 | 0 |
| (b) | MEHQ | 0 | 0 | 0 | 0 | 0 | 0 |
| | BHT | 0.19 | 4.9 | 0.15 | 1.18 | 0.15 | 0.23 |
| (c) | Acetone | 0 | 0 | 0 | 1180 | 0 | 0 |
| | Ethanol | 190 | 0 | 150 | 0 | 150 | 0 |
| (d) | CQ | 1.9 | 4.9 | 0 | 0 | 0 | 2.3 |
| | BAPO | 0 | 4.9 | 0 | 0 | 0 | 0 |
| | DMBE | 1.9 | 4.9 | 0 | 0 | 0 | 0 |
| | IPIFP | 0 | 0 | 0 | 0 | 0 | 0 |
| | DEPT | 0 | 0 | 0 | 0 | 0 | 0 |
| | BPO | 0 | 0 | 0 | 0 | 0 | 0 |
| | CHP | 0 | 0 | 0 | 0 | 0 | 2.3 |
| | TMBH | 0 | 0 | 0 | 0 | 0 | 0 |
| | VOA | 0 | 0 | 0 | 0 | 0 | 0 |
| | COA | 0 | 0 | 0 | 0 | 0 | 0 |
| | PTU | 0 | 0 | 0 | 0 | 0 | 0 |
| (e) | R-974 | 0 | 0 | 0 | 0 | 0 | 0 |

| Empty space rate (%) | | 80 | 80 | 90 | 20 | 90 | 90 |
|---|---|---|---|---|---|---|---|
| Production machine 1 | | | ∘ | | | | |
| Production machine 2 | | | | ∘ | ∘ | | ∘ |
| Production machine 3 | | ∘ | | | | ∘ | |
| Production machine 4 | | | | | | | |
| Mixing time required | | 1 | 2 | 1 | 1 | 1 | 15 |
| to reach homogeneity | | | | | | | |
| Viscosity of the final | | 5 | 6 | 1 | 1 | 1 | 849 |
| product mPa · s | | | | | | | |
| Yield | | ∘ | ∘ | ∘ | ∘ | ∘ | ∘ |
| Storage stability | | ∘ | ∘ | ∘ | ∘ | ∘ | ∘ |
| | | | Example 29 | Example 30 | Example 31 | Example 32 | Example 33 |
| | (a) | BisGMA | 1212 | 0 | 0 | 1080 | 0 |
| | | UDMA | 0 | 132 | 264 | 0 | 1050 |
| | | TEGDMA | 0 | 88 | 0 | 0 | 700 |
| | | GDMA | 808 | 0 | 176 | 720 | 0 |
| | | HEMA | 0 | 0 | 0 | 0 | 0 |
| | | MDP | 0 | 0 | 0 | 0 | 0 |
| | | MHPA | 0 | 0 | 0 | 0 | 0 |
| | | META | 0 | 0 | 0 | 0 | 0 |
| | (b) | MEHQ | 2.02 | 0 | 0.44 | 1.8 | 0 |
| | | BHT | 0 | 0.22 | 0 | 0 | 1.75 |
| | (c) | Acetone | 0 | 0 | 0 | 0 | 0 |
| | | Ethanol | 0 | 0 | 0 | 0 | 0 |
| | (d) | CQ | 20.2 | 2.2 | 4.4 | 18 | 0 |
| | | BAPO | 0 | 0 | 0 | 0 | 17.5 |
| | | DMBE | 20.2 | 0 | 4.4 | 0 | 0 |
| | | IPIFP | 0 | 0 | 0 | 0 | 0 |
| | | DEPT | 0 | 0 | 0 | 0 | 0 |
| | | BPO | 0 | 2.2 | 0 | 0 | 17.5 |
| | | CHP | 0 | 0 | 4.4 | 0 | 0 |
| | | TMBH | 20.2 | 0 | 0 | 18 | 0 |
| | | VOA | 0 | 0 | 0 | 0 | 0 |
| | | COA | 0 | 0 | 0 | 0 | 0 |
| | | PTU | 0 | 0 | 0 | 0 | 0 |
| | (e) | R-974 | 0 | 0 | 0 | 0 | 0 |

| | | | | | |
|---|---|---|---|---|---|
| Empty space rate (%) | 10 | 90 | 80 | 20 | 20 |
| Production machine 1 | ∘ | | | ∘ | |
| Production machine 2 | | | ∘ | | |
| Production machine 3 | | ∘ | | | ∘ |
| Production machine 4 | | | | | |
| Mixing time required | 15 | 13 | 9 | 13 | 10 |
| to reach homogeneity | | | | | |
| Viscosity of the final | 854 | 154 | 161 | 850 | 152 |
| product mPa · s | | | | | |
| Yield | ∘ | ∘ | ∘ | ∘ | ∘ |
| Storage stability | ∘ | ∘ | ∘ | ∘ | ∘ |

TABLE 4

| | | Comparative | Comparative | Comparative | Comparative | Comparative | Comparative |
|---|---|---|---|---|---|---|---|
| | | Example 1 | Example 2 | Example 3 | Example 4 | Example 5 | Example 6 |
| (a) | BisGMA | 12 | 0 | 12 | 0 | 17 | 0 |
| | UDMA | 0 | 1296 | 0 | 1356 | 0 | 1824 |
| | TEGDMA | 0 | 0 | 0 | 0 | 1 | 0 |
| | GDMA | 8 | 864 | 7 | 791 | 0 | 114 |
| | HEMA | 0 | 0 | 0 | 22.6 | 0 | 228 |
| | MDP | 0 | 0 | 1 | 0 | 0 | 0 |
| | MHPA | 0 | 0 | 0 | 0 | 2 | 0 |
| | META | 0 | 0 | 0 | 90.4 | 0 | 114 |
| (b) | MEHQ | 0.02 | 0 | 0.02 | 0 | 0 | 45.6 |
| | BHT | 0 | 2.16 | 0 | 2.26 | 0.6 | 0 |
| (c) | Acetone | 0 | 0 | 0 | 0 | 0 | 0 |
| | Ethanol | 0 | 0 | 0 | 0 | 0 | 0 |
| (d) | CQ | 0.2 | 21.6 | 0.2 | 22.6 | 0.2 | 22.8 |
| | BAPO | 0 | 0 | 0 | 0 | 0 | 0 |
| | DMBE | 0.2 | 21.6 | 0.2 | 22.6 | 0.2 | 22.8 |
| | IPIFP | 0 | 0 | 0 | 0 | 0 | 0 |
| | DEPT | 0.2 | 21.6 | 0.2 | 22.6 | 0 | 0 |
| | BPO | 0 | 0 | 0 | 0 | 0 | 0 |
| | CHP | 0 | 0 | 0 | 0 | 0 | 0 |
| | TMBH | 0 | 0 | 0 | 0 | 0 | 0 |
| | VOA | 0 | 0 | 0 | 0 | 0 | 0 |
| | COA | 0 | 0 | 0 | 0 | 0 | 0 |
| | PTU | 0 | 0 | 0 | 0 | 0 | 0 |
| (e) | R-974 | 0 | 0 | 0 | 0 | 0 | 0 |

| Empty space rate(%) | | 99 | 1 | 99 | 1 | 99 | 1 |
|---|---|---|---|---|---|---|---|
| Production machine 1 | | | | | ∘ | | |
| Production machine 2 | | ∘ | ∘ | ∘ | | ∘ | |
| Production machine 3 | | | | | | | ∘ |
| Production machine 4 | | | | | | | |
| Mixing time required | | 24< | 24< | 24< | 24< | 48< | 48< |
| to reach homogeneity | | | | | | | |
| Viscosity of the final | | — | — | — | — | — | — |
| product mPa · s | | | | | | | |
| Yield | | ∘ | ∘ | ∘ | ∘ | ∘ | ∘ |
| Storage stability | | x | x | x | x | x | x |
| | | | Comparative | Comparative | Comparative | Comparative | Comparative |
| | | | Example 7 | Example 8 | Example 9 | Example 10 | Example 11 |
| | (a) | BisGMA | 12 | 0 | 0 | 0 | 462 |
| | | UDMA | 0 | 432 | 0 | 0 | 0 |
| | | TEGDMA | 0 | 72 | 0 | 0 | 77 |
| | | GDMA | 2 | 0 | 0 | 0 | 0 |
| | | HEMA | 3 | 72 | 0 | 0 | 77 |
| | | MDP | 3 | 0 | 0.8 | 0 | 77 |
| | | MHPA | 0 | 72 | 0 | 116.8 | 0 |
| | | META | 0 | 72 | 0.2 | 29.2 | 77 |
| | (b) | MEHQ | 0 | 0 | 0 | 0 | 0 |
| | | BHT | 0.1 | 3.6 | 0.01 | 1.46 | 3.85 |
| | (c) | Acetone | 20 | 0 | 0 | 1460 | 770 |
| | | Ethanol | 0 | 1080 | 10 | 0 | 0 |
| | (d) | CQ | 0.2 | 7.2 | 0 | 0 | 7.7 |
| | | BAPO | 0 | 0 | 0 | 0 | 0 |
| | | DMBE | 0.2 | 7.2 | 0 | 0 | 7.7 |
| | | IPIFP | 0 | 0 | 0 | 0 | 0 |
| | | DEPT | 0.2 | 0 | 0 | 0 | 7.7 |
| | | BPO | 0 | 0 | 0 | 0 | 0 |
| | | CHP | 0 | 0 | 0 | 0 | 0 |
| | | TMBH | 0 | 0 | 0 | 0 | 0 |
| | | VOA | 0 | 0 | 0 | 0 | 0 |
| | | COA | 0 | 0 | 0 | 0 | 0 |
| | | PTU | 0 | 0 | 0 | 0 | 0 |
| | (e) | R-974 | 0 | 0 | 0 | 0 | 1386 |

| | | | | | |
|---|---|---|---|---|---|
| Empty space rate(%) | 98 | 1 | 99 | 1 | 20 |
| Production machine 1 | ∘ | | | | |
| Production machine 2 | | ∘ | ∘ | | ∘ |
| Production machine 3 | | | | ∘ | |
| Production machine 4 | | | | | |
| Mixing time required | 24< | 24< | 3< | 3< | 24< |
| to reach homogeneity | | | | | |
| Viscosity of the final | — | — | 1 | 1 | — |
| product mPa · s | | | | | |
| Yield | ∘ | ∘ | ∘ | ∘ | ∘ |
| Storage stability | x | x | x | x | x |

TABLE 5

| | | Comparative | Comparative | Comparative | Comparative | Comparative |
|---|---|---|---|---|---|---|
| | | Example 12 | Example 13 | Example 14 | Example 15 | Example 16 |
| (a) | BisGMA | 0 | 0 | 12 | 0 | 672 |
| | UDMA | 114 | 343 | 0 | 1296 | 0 |
| | TEGDMA | 0 | 49 | 0 | 0 | 448 |
| | GDMA | 19 | 0 | 8 | 864 | 0 |
| | HEMA | 19 | 49 | 0 | 0 | 0 |
| | MDP | 0 | 0 | 0 | 0 | 0 |
| | MHPA | 19 | 0 | 0 | 0 | 0 |
| | META | 19 | 49 | 0 | 0 | 0 |
| (b) | MEHQ | 0 | 0 | 0.02 | 0 | 1.12 |
| | BHT | 1.9 | 4.9 | 0 | 2.16 | 0 |
| (c) | Acetone | 0 | 0 | 0 | 0 | 0 |
| | Ethanol | 190 | 0 | 0 | 0 | 0 |
| (d) | CQ | 1.9 | 4.9 | 0 | 21.6 | 11.2 |
| | BAPO | 0 | 4.9 | 0.2 | 0 | 0 |
| | DMBE | 1.9 | 4.9 | 0 | 21.6 | 11.2 |
| | IPIFP | 0 | 0 | 0 | 0 | 0 |
| | DEPT | 0 | 0 | 0 | 0 | 11.2 |
| | BPO | 0 | 0 | 0.2 | 0 | 0 |
| | CHP | 0 | 0 | 0 | 0 | 0 |
| | TMBH | 0 | 0 | 0 | 21.6 | 0 |
| | VOA | 0 | 0 | 0 | 0 | 0 |
| | COA | 0 | 0 | 0 | 0 | 0 |
| | PTU | 0 | 0 | 0 | 0 | 0 |
| (e) | R-974 | 190 | 490 | 0 | 0 | 0 |
| Empty space rate(%) | | 80 | 80 | 99 | 1 | 50 |
| Production machine 1 | | | ∘ | | | |
| Production machine 2 | | | | | ∘ | |
| Production machine 3 | | ∘ | | ∘ | | |
| Production machine 4 | | | | | | ∘ |
| Mixing time required | | 24< | 24< | 24< | 24< | 12 |
| to reach homogeneity | | | | | | |
| Viscosity of the final | | — | — | — | — | 802 |
| product mPa · s | | | | | | |
| Yield | | ∘ | ∘ | ∘ | ∘ | x |
| Storage stability | | x | x | x | x | ∘ |
| | | Comparative | Comparative | Comparative | Comparative | Comparative |
| | | Example 17 | Example 18 | Example 19 | Example 20 | Example 21 |
| (a) | BisGMA | 672 | 0 | 0 | 0 | 672 |
| | UDMA | 0 | 990 | 254.8 | 0 | 0 |
| | TEGDMA | 392 | 55 | 36.4 | 0 | 448 |
| | GDMA | 0 | 55 | 0 | 0 | 0 |
| | HEMA | 0 | 0 | 36.4 | 0 | 0 |
| | MDP | 56 | 0 | 0 | 59.2 | 0 |
| | MHPA | 0 | 0 | 0 | 0 | 0 |
| | META | 0 | 0 | 36.4 | 14.8 | 0 |
| (b) | MEHQ | 1.12 | 0 | 0 | 0 | 1.12 |
| | BHT | 0 | 22 | 1.82 | 0.74 | 0 |
| (c) | Acetone | 0 | 0 | 546 | 0 | 0 |
| | Ethanol | 0 | 0 | 0 | 740 | 0 |
| (d) | CQ | 11.2 | 11 | 3.64 | 0 | 11.2 |
| | BAPO | 0 | 0 | 3.64 | 0 | 0 |
| | DMBE | 11.2 | 11 | 3.64 | 0 | 11.2 |
| | IPIFP | 0 | 0 | 0 | 0 | 0 |
| | DEPT | 11.2 | 0 | 0 | 0 | 0 |
| | BPO | 0 | 0 | 0 | 0 | 0 |
| | CHP | 0 | 0 | 0 | 0 | 11.2 |
| | TMBH | 0 | 0 | 0 | 0 | 0 |
| | VOA | 0 | 0 | 0 | 0 | 0 |
| | COA | 0 | 0 | 0 | 0 | 0 |
| | PTU | 0 | 0 | 0 | 0 | 0 |
| (e) | R-974 | 0 | 0 | 0 | 0 | 0 |

| | | | | | |
|---|---|---|---|---|---|
| Empty space rate(%) | 50 | 50 | 50 | 50 | 50 |
| Production machine 1 | | | | | |
| Production machine 2 | | | | | |
| Production machine 3 | | | | | |
| Production machine 4 | ∘ | ∘ | ∘ | ∘ | ∘ |
| Mixing time required | 14 | 26 | 4 | 1 | 15 |
| to reach homogeneity | | | | | |
| Viscosity of the final | 802 | 7960 | 803 | 1 | 803 |
| product mPa · s | | | | | |
| Yield | x | x | x | x | x |
| Storage stability | ∘ | ∘ | ∘ | ∘ | ∘ |

Examples 1 to 33 demonstrated that the mixtures obtained had excellent yields and excellent storage stability. The mixing time required to homogenize the mixture was shorter when the empty space rate of the container was 20 to 80% than that when it was 10 to 90%. On the other hand, Comparative Examples 1 to 10 and Comparative Examples 14 and 15 demonstrated that the mixing time required to homogenize the mixture was longer or rendered homogenization difficult, and the storage stability of the mixtures obtained was poor. The empty space rate that was less than 10% or more than 90% reduced the mixing efficiency and made it difficult to obtain a homogenous mixture, as a result of which the storage stability is considered to have been adversely affected.

In Comparative Examples 11 to 13, the (e) filler was contained in the compounded components, making it difficult for the (e) filler to be homogeneously mixed in the mixture, and the storage stability of the mixtures obtained was poor. In order to homogeneously disperse the (e) filler in the dental composition, it is considered to be favorable to use the production machine equipped with a stirring blade and a container.

Comparative Examples 16 to 21 demonstrated that the homogeneous mixture was obtained as in the case of the Examples, however, the production machine using the stirring blade was used, resulting in the poor yield of the mixture.

### INDUSTRIAL APPLICABILITY

The present invention can be industrially used in the dental field as a method for producing dental polymerizable compositions such as dental dentin adhesives.

1. A method for producing a dental polymerizable composition (β) or a dental polymerizable composition intermediate (γ), which is substantially free of a filler, using a production machine that is substantially not equipped with a stirring blade and equipped with a container, the method comprising the steps of: disposing a composition (α) comprising (a) a polymerizable monomer and (b) a polymerization inhibitor in the container such that an empty space rate of the container is 10 to 90%; and allowing the container to operate.

2. A method for producing a dental polymerizable composition (β) or a dental polymerizable composition intermediate (γ), which is substantially free of a filler, using a production machine that is substantially not equipped with a stirring blade and equipped with a container, the method comprising the steps of: disposing a composition (α) comprising (a) a polymerizable monomer and (b) a polymerization inhibitor in the container such that an empty space rate of the container is 10 to 90%; heating the container to between 3° and 70° C.; and allowing the container to operate.

3. The method for producing a dental polymerizable composition (β) or a dental polymerizable composition intermediate (γ) according to claim 1 or 2, wherein in the production machine that is substantially not equipped with a stirring blade and equipped with a container, the container operates in one or more modes selected from rotation, hyperbolic motion, or vibration.

4. The method for producing a dental polymerizable composition (β) or a dental polymerizable composition intermediate (γ) according to claim 1 or 2, wherein in the production machine that is substantially not equipped with a stirring blade and equipped with a container, the container operates in one or more modes selected from rotation at a speed of 60 min⁻¹ or less, hyperbolic motion at a speed of 50 min⁻¹ or less, or vibration at a speed of 400 min⁻¹ or less.

5. The method for producing a dental polymerizable composition (β) or a dental polymerizable composition intermediate (γ) according to claim 1 or 2, wherein the empty space rate of the container is 20 to 80%.

6. The method for producing a dental polymerizable composition (β) or a dental polymerizable composition intermediate (γ) according to claim 1 or 2, wherein the composition (α) comprises (c) an organic solvent.
